(19) United States
(12) Patent Application Publication
(10) Pub. No.: US 2024/0279754 A1
(43) Pub. Date: Aug. 22, 2024

(54) RAPID, MULTIPLEX SYSTEM FOR DIRECTLY VISUALIZING AND QUANTIFYING DRUG RESISTANCE IN SINGLE VIRIONS AND VIRION POPULATIONS AND USES THEREOF

(71) Applicants: UNIVERSITY OF MARYLAND, BALTIMORE, Baltimore, MD (US); ISS Inc., Champaign, IL (US)

(72) Inventors: Krishanu RAY, Ellicott City, MD (US); Anthony L. DEVICO, Alexandria, VA (US); Beniamino BARBIERI, Champaign, IL (US); Yuansheng SUN, Champaign, IL (US)

(21) Appl. No.: 18/443,665

(22) Filed: Feb. 16, 2024

Related U.S. Application Data
(60) Provisional application No. 63/485,597, filed on Feb. 17, 2023.

(51) Int. Cl. C12Q 1/70; C12Q 1/02; C12Q 1/6818; G01N 21/64; G16B 40/10; G16H 20/17

(52) U.S. Cl. CPC C12Q 1/703; C12Q 1/025; C12Q 1/6818; G01N 21/6486; G16B 40/10; G16H 20/17

(57) ABSTRACT

A fluorescence-based analytical platform comprising methods, software and instrumentation that accomplishes direct, rapid, high throughput, multiplex, and quantitative determinations of virion populations, in human or animals or experimental fluids, that are impacted by, or escape from, interactions with antiviral drugs. The platform can be used to advance bnAb resistance detection capacities to support numerous bnAb clinical trial activities, from screening volunteers to tailoring subject-specific bnAb combinations for treatment or cure.

### CROSS REFERENCE TO RELATED APPLICATIONS

This application is filed under the provisions of 35 U.S.C. § 111(a) and claims priority to U.S. Provisional Patent Application No. 63/485,597 filed on Feb. 17, 2023 in the name of Krishanu Ray, et al., and entitled “RAPID, MULTIPLEX SYSTEM FOR DIRECTLY VISUALIZING AND QUANTIFYING DRUG RESISTANCE IN SINGLE VIRIONS AND VIRION POPULATIONS AND USES THEREOF,” which is hereby incorporated by reference herein in its entirety.

### STATEMENT OF FEDERALLY SPONSORED RESEARCH

This invention was made with government support under Grant Numbers GM117836, AI150447, AI172487 and AI176561 awarded by the National Institutes of Health. The government has certain rights in the invention.

### FIELD

The present invention relates to a fluorescence-based platform that is used to quantitatively analyze natural virion populations in a subject to determine sensitivity to, or escape from, interactions with antiviral drugs alone or in combination.

### DESCRIPTION OF THE RELATED ART

The discovery and isolation of broadly neutralizing antibodies (bnAbs) targeting the HIV envelope has opened up new possibilities in the quest for long-acting HIV/AIDS treatment, functional cure and/or prevention. Among the advantages of bnAbs are their ability to be formulated as long-acting agents and to trigger cell-mediated immune defense mechanisms, such as antibody-dependent cellular cytotoxicity (ADCC). However, as with most small-molecule therapeutics, treatment with monotherapy can lead to resistance.

Ongoing exploration of this opportunity has included multiple clinical trials with several bnAbs. The emerging lesson is that gaps in bnAb coverage of viral Env diversity in people living with HIV present multiple hurdles for achieving clinical efficacy. From the virological perspective, Env diversity almost certainly explains why the bnAb trials conducted to date have repeatedly seen only limited reductions in transmission risk or transient reductions in viremia followed by rebound of resistant strains. Specifically, the uninformed enrollment of people living with HIV harboring bnAb-resistant, replication competent, and transmissible virus variants in plasma and/or reservoir compartments negatively skews any estimates of virologic suppression, confounds the definition of clinical use parameters, and obscures potential paths to using bnAbs as HIV countermeasures.

Going forward, tests that provide prospective knowledge of subjects' virus sensitivity/resistance would vastly improve clinical trial studies of bnAbs and other drugs in prevention and therapy. To enable efficient screening of potential volunteers for therapy trials, such tests should generate data in ≤1-2 weeks. In addition, they should be sensitive enough to inform the nature of breakthrough viruses detected in prevention trials. The ideal assays would also facilitate tailoring bnAb combinations to a subject's variants (analogous to tests of phenotypic drug resistance and tropism) and possess aspects amenable to commercial development for routine clinical applications. Future bnAb resistance tests must also accommodate the growing interest in mitigating escape via administering combinations of bnAbs against collections of different epitopes. Triple combinations of bnAb classes are thought to strike the best balance between feasibility and efficacy. Therefore, analytical methods must reveal not only the extent of virus coverage by each bnAb but the nature of aggregate coverage, which also impacts efficacy. When bnAbs in a combination each bind a subset of a virus swarm, escape from only one antibody could allow unbridled replication with no fallback protection. Conversely, this risk may be mitigated when two or more bnAbs concurrently/redundantly bind virions or Env trimers in major subpopulations of a swarm [K. Wagh et al., 2016; F. Klein et al., 2012; R. Kong et al., 2015; K. Wagh et al., 2018].

Current approaches to screen for bnAb resistance in clinical settings, although clearly informative, do not meet these needs. The main hindrance is dependence on viral Env gene sequencing, which, although informative, is labor intensive, technically complex, and requires a 4-6 week turn-around time from sample collection to results. Envs suspected of resistance must be incorporated in pseudoviruses and retested against the bnAb of interest to directly demonstrate escape from recognition. In some perplexing cases, escape variants are not identified, as the recovered virus Env sequences remain sensitive to the test bnAb [P. Mendoza et al., 2018; R. Schoofs et al., 2016]. Another caveat is that structured treatment interruption (STI) trials involve bnAbs administration to antiretroviral therapy (ART)-treated subjects, who have little or no circulating HIV RNA to assess. One way around this problem is to pursue Env sequences from the HIV DNA reservoir; but this is also a lengthy effort that could delay study enrollment. Further, HIV proviral DNA sequences may produce misleading results as they poorly represent replication competent genomes that might establish rebound viremia. An alternative approach is to utilize an outgrowth system in which a trial participant's cells are cultured with exogenous target cells to propagate replicating viruses, which are then subjected to sequence/neutralization analyses. These approaches add more time to the analytical process.

Currently there are no platforms or analytical systems, clinically applicable or otherwise, that accomplish direct, rapid, high throughput, multiplex, quantitative analyses of natural virion populations, in human, animal or experimental fluids, to determine sensitivity to, or escape from, interactions with antiviral drugs (e.g., bnAbs) alone or in combination. Accordingly, it is the objective of the present disclosure to introduce new clinical platforms and associated laboratory instruments that directly, rapidly, and concurrently detect resistance or sensitivity to multiple bnAbs in samples or cell cultures from viremic subjects and people on ART.

### SUMMARY

In one aspect, method for quantifying a fraction of virions in a fluid sample that are sensitive to at least one drug is described, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the fraction of virions in the fluid sample that are sensitive to the at least one drug by calculating an Instantaneous Inhibition Potential (IIP) value of the at least one drug,
   wherein the IIP is an indication of the fractions of virions in the fluid sample that are sensitive to the at least one drug.

In another aspect, a method of calculating an Instantaneous Inhibition Potential (IIP) value of at least one drug in a fluid sample is described, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the IIP value of the at least one drug in the fluid sample, wherein the IIP value is the number of log 10 infections reduced by the at least one drug.

In yet another aspect, a method of identifying if at least one drug will substantially eliminate virus replication and/or suppress viral load to levels below detection in a subject is described, said method comprising:
calculating the IIP value of the at least one drug using a method described herein, wherein if the calculated IIP value is greater than a threshold IIP determined for a known, efficacious drug, the virions are sensitive to the at least one drug, and said drug will likely substantially eliminate virus replication and/or suppress viral load to levels below detection.

In still another aspect, a method of identifying, prior to treatment, at least one drug that extensively covers wild-type virus populations in a subject is described, said method comprising:
calculating the IIP value of the at least one drug using a method described herein, wherein if the calculated IIP value is greater than a threshold IIP determined for a known, efficacious drug, the virions are sensitive to the at least one drug, and said at least one drug will likely cover the virus in said subject.

In yet another aspect, a method of identifying emerging endogenous bnAb resistance after exogenous bnAb treatment of a subject is described, said method comprising:
calculating the IIP value, in temporal samples, of the bnAb using a method described herein, wherein if the IIP value decreases in the temporal samples, the virions are becoming resistant to the bnAb.

In still another aspect, an apparatus for detecting interacting drug-virion bursts at a single virion level in an observation volume is described, said apparatus comprising:
a multi-laser launcher;
a plate-based moving stage;
a multi-channel single molecule spectrophotometer; and
a computing device.

In some embodiments, the spectrophotometer comprises sample locations selected from cover slides with sealed silicone chambers, chambered slides, or glass-bottom 96- or 384-well plates. In some embodiments, drug-virion interactions are detected using fluorescence correlation spectrometry (FCS) using up to four excitation wavelengths. In some embodiments, the excitation wavelengths are 488, 561, 635 and 730 nm.

In some embodiments, the fluorophores are excited through a water objective. In some embodiments, the computing device controls the apparatus, performs data acquisition and processes the data. In some embodiments, the computing device comprises pattern recognition software and algorithms, which separate interacting drug-virion bursts from non-interacting drug signals and background noise and reconstructs the total number of interacting drug-virions present in the observation volume. In some embodiments, single drug interactions to virions are identified by signals in one dedicated detector. In some embodiments, concurrent interactions of multiple drugs to virions are identified by cross-correlation analyses of multi-channel signals. In some embodiments, the interacting of two or more drugs to a single protein on a virion surface is detected using fluorescent resonant energy transfer (FRET).

In yet another aspect, method of quantifying a fraction of virions in a fluid sample that are sensitive to at least one drug using an apparatus is described, wherein the apparatus comprises a multi-laser launcher; a plate-based moving stage; a multi-channel single molecule spectrophotometer; and a computing device, said method comprising:
mixing the fluid sample with at least one fluorescent-labeled drug conjugate to encourage drug-virion interactions in the mixture;
introducing the mixture to a sample location in the spectrophotometer;
using the multi-laser launcher to excite the fluorophores in the mixture;
using the spectrophotometer to detect interacting drug-virion bursts at a single virion level in an observation volume;
counting bursts in at least one detector channel using an algorithm;
determining a total population size of virions in the fluid sample using an algorithm; and
calculating the fraction of virions in the fluid sample that are sensitive to the at least one drug by calculating an IIP value of the at least one drug as described herein, wherein the IIP is an indication of the fractions of virions in the fluid sample that are sensitive to the at least one drug.

In yet another aspect, a method of calculating an IIP value of at least one drug in a fluid sample using an apparatus is described, wherein the apparatus comprises a multi-laser launcher; a plate-based moving stage; a multi-channel single molecule spectrophotometer; and a computing device, said method comprising:
mixing the fluid sample with at least one fluorescent-labeled drug conjugate to encourage drug-virion interactions in the mixture;
introducing the mixture to a sample location in the spectrophotometer;
using the multi-laser launcher to excite the fluorophores in the mixture;
using the spectrophotometer to detect interacting drug-virion bursts at a single virion level in an observation volume;
counting bursts in at least one detector channel using an algorithm;
determining a total population size of virions in the fluid sample; and
calculating the IIP value of the at least one drug in the fluid sample, wherein the IIP value is the number of log 10 infections reduced by the at least one drug.

In another aspect, a method of calculating an Instantaneous Inhibition Potential (IIP) value of a sample using computational software/algorithms is described, said method comprising:
recording of fluorescence data of the sample at least one dye-specific detection channel;
recording time-stamped photons by a data-acquisition card for the sample at a specific spectral wavelength; generating fluorescent signal intensity-time traces for each dye-specific detection channel;
using an algorithm to determine signal threshold, signal background, signal dwell time, and diffusion-rate of the signal;
using an algorithm to identify bursts from time-tagged data by fitting target-defined diffusion time and intensity parameters;
using a filter function and threshold to clean background noise;
using a filter function to sort bursts based on diffusion rates;
executing an autocorrelation and fitting routine to validate selection of drug-bound target bursts;
determining the number of bursts representing selected targets seen during an assay run time;
optionally analyzing cross-correlated or FRET signals; and
calculating the IIP of the drug.

Other aspects, features and advantages of the invention will be more fully apparent from the ensuing disclosure and appended claims.

### BRIEF DESCRIPTION OF THE DRAWINGS

FIG. 1. Detection capacities of an FCS-based bnAb resistance/sensitivity assay. The illustration depicts three different bnAb types, each conjugated to a different dye (red, green, or blue) that enables cross-correlated signal detection. In a single test of a sample, the numbers of virions in each situation (bound to one or more bnAb types) can be detected and counted and compared to the size of the total sample population.

FIG. 2. Two modes of FCS based detection assays for virion susceptibility to HIV bnAbs in plasma and culture fluids.

FIG. 3A. Intensity-time trace for the interaction of bnAb N49P7-A647 with HIV+human plasma (black-trace). Intensity-time trace of N49P7-A647 (1 nM) alone is shown in green-trace. The threshold for detecting fluorescently labeled bnAb bound virion bursts is set at two-times above the highest intensity from the fluorescent bnAb alone. Bursts from N49P7-A647 bound virions (red-trace) are separated from the experimental data (black-trace) using thresholding.

FIG. 3B. Autocorrelation plots are fit to each trace from the FIG. 3A. Green and red traces are fitted only with single diffusion coefficients of 55 and 6 μm²/sec, corresponding to bnAb N49P7 and N49P7 bound plasma virion, respectively. Red burst counts reflect bnAb bound virions, which are counted.

FIG. 4A. Procedure for the FCS co-culture mode to detect bnAb sensitivity or resistance using healthy CD4+ cells or MOLT-4 cells for virus propagation (Figure adapted from G. M. Laird, 2016 and J. C. Lorenzi, 2016).

FIG. 4B. HIV virions concentrated from culture supernatants were assayed by FCS burst counting to determine numbers/ml bound to PGT121, N49P7 or CAP256-VRC26.25. The dashed line represents total virion concentration determined by RT-qPCR. Subtractions of values solves for the fraction of the population not bound by bnAb. All experiments were repeated three times, and average values are shown. Error bars indicate standard deviations from triplicate measurements. Control tests with nonspecific IgG1 produced signals with diffusion rates corresponding only to free antibody. Experiments were repeated two times with similar results.

FIG. 5A. Concentration or inactivation processes do not alter relative bnAb binding efficiencies in FCS. Percent of bnAbs PGT121, N49P7, CAP256-VRC26.25 bound to virions generated in the outgrowth co-culture assay mode using MOLT-4 cells. bnAb binding patterns before and after PEG-it™ concentration are shown. The negative control was irrelevant human IgG1.

FIG. 5B. Percent binding of bnAbs PGT121, N49P7 or CAP256-VRC26.25 to HIV BaL infectious molecular clone viruses. NEM inactivation of bnAb-virion sample did not change the percent bound population. Data were collected from triplicate tests; average values are shown. Error bars=standard deviation.

FIG. 6. Schematics of a benchtop instrument for performing FCS-based bnAb-virion binding assay in clinical settings.

FIG. 7. Flowchart showing the steps of data analysis using the software and algorithm described herein with regards to the fluorescence-based platform used to quantitatively analyze natural virion populations in a subject.

FIG. 8A. HIV virions concentrated from culture supernatants was assayed by FCS burst counting to determine numbers/ml bound to mAbs PGT121, N49P7 or CAP256-VRC26.25 for Subject 1. The dashed line represents total virion concentration determined by RT-qPCR. Subtractions of values solves for the fraction of the population not bound by bnAb.

FIG. 8B. Percentage of bnAb bound HIV-1 population for subject 1.]

FIG. 8C. Correlation between % binding versus % neutralization was determined for Subject 1.

FIG. 8D. HIV virions concentrated from culture supernatants was assayed by FCS burst counting to determine numbers/ml bound to mAbs PGT121, N49P7 or CAP256-VRC26.25 for Subject 2. The dashed line represents total virion concentration determined by RT-qPCR. Subtractions of values solves for the fraction of the population not bound by bnAb.

FIG. 8E. Percentage of bnAb bound HIV-1 population for subject 2.

FIG. 8F. HIV virions concentrated from culture supernatants was assayed by FCS burst counting to determine numbers/ml bound to mAbs PGT121, N49P7 or CAP256-VRC26.25 for Subject 3. The dashed line represents total virion concentration determined by RT-qPCR. Subtractions of values solves for the fraction of the population not bound by bnAb.

FIG. 8G. Percentage of bNAb bound HIV-1 population for subject 3.

FIG. 9A. CAP256-VRC26.25-A488 (anti-V2 apex)+PGT121-A594 (anti-V3 glycan) binding to plasma virions by cross-correlation analyses.

FIG. 9B. PGT121-A594+N49P7-A647 (anti-CD4bs) binding to plasma virions by cross-correlation analyses.

FIG. 10. Potential mAb binding situations distinguishable by FRET and dual (three-color) FRET.

FIG. 11A. Dual FRET analyses of triple mAb combinations reacted with plasma virions.

FIG. 11B. Dual FRET analyses of triple mAb combinations reacted with plasma virions.

FIG. 11C. Autocorrelation plots of the donor PGDM1400-A488 with plasma virions at concentrations of 5 nM. Solid lines represent fitting of experimental data. The autocorrelation plot fits a two species diffusion model.

FIG. 11D. Autocorrelation plots of the acceptor PGT121-A594 with plasma virions at concentrations of 5 nM. Solid lines represent fitting of experimental data. The autocorrelation plot fits only a single species of 6 μm²/sec.

FIG. 11E. Autocorrelation plots of the acceptor N49P7-A647 with plasma virions at concentrations of 5 nM. Solid lines represent fitting of experimental data. The autocorrelation plot fits only a single species of 6 μm²/sec.

### DETAILED DESCRIPTION, AND PREFERRED EMBODIMENTS THEREOF

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Sample,” “test sample,” “fluid sample,” and “test fluid” may be used interchangeably herein and refer to blood, plasma, serum, mucosal fluids, bronchial lavage samples, or cell cultures.

“Subject” and “patient” as used herein interchangeably refers to any vertebrate, including, but not limited to, an animal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human. In some embodiments, the subject is a human.

As described herein, “wild-type virus population(s)” or “virus population(s)” or “sample population(s)” include those extracted from plasma or infected cells.

As defined herein, reference to a “drug” or “antiviral drug” includes an antibody, an antiviral drug, e.g., a small molecule, a combination of antibodies, a combination of at least one antibody and at least one antiviral drug, or a combination of antiviral drugs. In some embodiments, the drug includes a broadly neutralizing antibody (bnAb) or a combination of bnAbs. In some embodiments, the drug includes a small molecule drug or a combination of small molecule drugs. In some embodiments, the drug includes a microbe or a combination of microbes. In some embodiments, the drug includes at least one antibody, e.g., bnAb, and at least one small molecule.

As used herein, the term “microorganism” includes bacteria, viruses, and fungi.

As used herein, the term “virus” can include, but is not limited to, retroviruses, influenza viruses, and sarbecoviruses/coronaviruses.

As used herein, “interacting,” “covering” and binding are intended to be synonymous.

As used herein, a “system” refers to a plurality of real and/or abstract elements operating together for a common purpose. In some embodiments, a “system” is an integrated assemblage of hardware and/or software elements. In some embodiments, each component of the system interacts with one or more other elements and/or is related to one or more other elements. In some embodiments, a system refers to a combination of components and software for controlling and directing methods.

As defined herein, “unmanipulated” means that nothing is added to, nor extracted from, the sample prior to analysis.

As defined herein, “a threshold determined for a known, efficacious drug” corresponds to the IIP threshold of a known-to-be-clinically-efficacious drug (e.g., determined using other methods) determined using the fluorescence-based platform described herein, to establish its IIP threshold. New drugs, or drugs new to the particular subject's sample, can then be tested, relative to this IIP threshold to see if they have a better (i.e., more sensitive) or worse (i.e., more resistant) IIP than the known-to-be-clinically-efficacious drug.

As defined herein, the terms “fluorescent label”, “fluorescent dye”, and “fluorophore” refer to moieties that absorb light energy at a defined excitation wavelength and emit light energy at a different wavelength. Examples of fluorescent labels include, but are not limited to: ALEXA FLUOR® dyes (ALEXA FLUOR® 488, 568, 594, 647, 700, 750), BODIPY® dyes, Carboxyrhodamine 6G, carboxy-X-rhodamine (ROX), Cyanine dyes (Cy3, Cy5, Cy3.5, Cy5.5), JANELIA FLUOR® Dyes (JF525, JF549, JF585, JF635, JF646, JF669), 4′,5′-Dichloro-2′,7′-dimethoxy-fluorescein, Fluorescein, FAM, Green Fluorescent Protein (GFP), Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), TEXAS RED®, Texas Red-X, and any combination thereof. In some embodiments, ALEXA FLUOR® dyes are used for making conjugates given their higher extinction coefficients, brightness and stability, e.g., dye-conjugated sCD4.

Broadly, the present disclosure relates to rapid and facile neutralization resistance tests based on direct detection and quantification of drug-microorganism binding within the native microorganism population of people or animals. Although hereinafter the application will refer to viruses as the microorganisms, it should be appreciated by the person skilled in the art that the methods and systems described herein can be applied to other microorganisms, e.g., determining bacterial drug sensitivity versus resistance. Accordingly, in some embodiments, the present disclosure relates to rapid and facile neutralization resistance tests based on direct detection and quantification of drug-virus binding within the native virus population of people or animals. Without being bound by theory, it is believed that potent drug, e.g., bnAb, neutralization ensues because of virion binding, while escape variants are by nature poorly (or are not) immunoreactive. Escape-prone situations are indicated when bnAbs bind (or “cover”) only a fraction of a virus population, leaving the remainder unrecognized and/or when each bnAb in a combination binds only a specific subset of the population. The more escape-resistant situations occur when multiple bnAb types concurrently react with a major fraction of virions in a sample population. For example, potential volunteers for STI/ATI+bnAb combination trials could be screened using one multiplex laboratory test that generates bnAb binding data in a practical timeframe (<2 weeks), without demanding more complicated, expensive, and time-consuming neutralization assays and genetic sequencing. Proactively knowing in real time that some potential volunteers harbor virus subsets that are not covered or bound by the bnAbs could guide trial enrollment and interpretation, e.g. such persons could be excluded as they are at high risk of virus rebound.

A fluorescence-based analytical platform can be used, wherein the platform comprises methods, software and instrumentation that accomplishes direct, rapid, high throughput, multiplex, quantitative determinations of virion populations, in a sample from a subject, wherein the virion population can be impacted by or can escape from interactions with antiviral drugs, whether the drugs are applied alone or in combination. The platform can be used to quantify the fractions of virions in sample populations that react with (i.e., are sensitive to) or escape from (i.e., are resistant to) a drug, with the result of revealing levels of drug sensitivity or resistance by determining an IIP value. In some embodiments, the sample population comprises fluids that are unmanipulated. In some embodiments, the sample population comprises fluids that have been prepared, e.g., extracted from a plasma or infected cells. This platform holds particular utility in the realm of testing drugs, e.g., bnAbs, against highly mutable and variable viral strains (e.g., retroviruses, sarbecoviruses/coronaviruses, influenza viruses). In some embodiments, confocal fluorescence correlation spectroscopy (FCS) techniques can be used for detecting drug binding at the single virion level. Using fluorescent labeled drugs, e.g., bnAbs, in FCS, it is possible to simultaneously count, in one multiplex assay, the numbers of virions in a virus population that are bound by one or more bnAb types or avoid recognition altogether because they are not covered. Advantageously, virions concurrently bound to multiple bnAb types can also be counted by tracking cross-correlated binding signals.

With regards to the units of detection reported, for the purposes of the present description, the viruses that are not substantially bound by the at least one drug (e.g., bnAb(s)) are considered “resistant” to treatment by said at least one drug. Therefore, the detection of unbound viruses signals drug resistance in a test sample. The methods described herein provide a unique ability to determine an Instantaneous Inhibition Potential (IIP) of a drug. IIP is an important metric, as it concerns the immediate, unalloyed interaction of the drug with its viral target (e.g., bnAb binding). Viewed simply, IIP is the number of log 10 infections reduced by a drug, e.g., an antiviral agent, at a selected test concentration, or log (fₐ/fᵤ), where fᵤ is the fraction of targets unaffected (unbound) by the drug and fₐ is the fraction rendered noninfectious (via drug binding). If one drug is tested or used, the calculated IIP relates to the effect of the one drug. If a combination of drugs are tested or used, the calculated IIP relates to the effect of the combination of drugs. In some embodiments, fₐ/fᵤ fractions are directly detected, which is an advance over the prior art, which estimated IIP mathematically using data from specialized in vitro test formats and the median effect equation. It should be appreciated that the exact numerical value of fₐ/fᵤ, and hence log(fₐ/fᵤ), will vary among viruses and among drugs, as readily determined by the person skilled in the art. For example, based on clinical data in HIV infection, successful antiretroviral therapy requires a threshold IIP value≥of 5-8. In other words, if FCS as described herein was used to test a drug at a given concentration against a sample comprising a virus and the IIP was determined to be 3, it could be concluded that the sample viruses have a level of resistance unacceptable for use of the drug in the clinic because of the risk of rebound. Accordingly, in some embodiments, a IIP value, or log(fₐ/fᵤ) value, <about 5 represents a sample comprising a virus that is overly resistant to the drug while a IIP value>about 5 represents a sample comprising a virus that is sensitive to the drug. In some embodiments, a IIP value, or log(fₐ/fᵤ) value, <about 6 represents a sample comprising a virus that is overly resistant to the drug while a IIP value>about 6 represents a sample comprising a virus that is sensitive to the drug. In some embodiments, a IIP value, or log(fₐ/fᵤ) value, <about 7 represents a sample comprising a virus that is overly resistant to the drug while a IIP value>about 7 represents a sample comprising a virus that is sensitive to the drug. In some embodiments, a IIP value, or log(fₐ/fᵤ) value, <about 8 represents a sample comprising a virus that is overly resistant to the drug while a IIP value>about 8 represents a sample comprising a virus that is sensitive to the drug. It should be appreciated by the person skilled in the art that the units of detection reported can include proportions, fractions, and percent.

As indicated, the exact numerical value of an IIP will vary among viruses and among drugs, as readily determined by the person skilled in the art. Accordingly, in some embodiments, a IIP value, or log(fₐ/fᵤ) value, <about 3 represents a sample comprising a virus that is overly resistant to the drug while a IIP value>about 3 represents a sample comprising a virus that is sensitive to the drug. In some embodiments, a IIP value, or log(fₐ/fᵤ) value, <about 4 represents a sample comprising a virus that is overly resistant to the drug while a IIP value>about 4 represents a sample comprising a virus that is sensitive to the drug.

The platform described herein is useful as a rapid, economical, multiplex clinical test for a sample that determines the presence and proportions, or IIP value, of virus subpopulations in the sample that are covered (or ignored) by one or more drugs or bnAbs (e.g., in a triple class combination) and thus reliably and sensitively identifies people living with a virus, e.g., HIV, who harbor resistant viruses risking rebound under drug or bnAb treatment. The platform also permits for the characterization of the nature of breakthrough infections in prevention trials.

Advantageously, using the platform described herein, a sample from viremic individuals can be rapidly screened to inform which drugs/combinations are most likely to be efficacious in clinical therapy/prevention settings. In addition, advantageously, the platform and methods described herein are not limited to the HIV virus, but can be used for other retroviruses, influenza viruses, and sarbecoviruses/coronaviruses. Further, the FCS platform described herein requires no buffers or reagent replacement or replenishment during the assay. As indicated hereinabove, the platform and methods described herein can also be used for other microorganisms, as understood by the person skilled in the art.

As indicated, innovative applications of confocal FCS methods for detecting drug, e.g., antibody, interactions at the single virion level can be used. The fundamental principle behind FCS is that random diffusion of a fluorophore (i.e., labeled probe) results in time-dependent fluorescence intensity fluctuations in a focal volume (observed by confocal microscopy), representing diffusion rates that are inversely proportional to the size of the object. Thus, FCS detects the binding of fluorescently tagged antibodies to free virions as a function of changes in diffusion rate: a virion bound antibody exhibits an 8-fold-lower diffusion rate than does free antibody. The fraction of a rapidly diffusing species (free antibody) that becomes more slowly diffusing (bound antibody) reveals the occurrence and efficiency of virus-antibody binding as governed by recognition of a cognate epitope. FCS data is collected with both single virions and single molecules continuously in solution at physiologically relevant concentrations with no need for direct protein engineering, chemical modifications, or other manipulations of target [M Mengistu et al., 2015; K. Ray et al., 2019; K. Ray et al., 2014; P. Agrawal, 2019]. Importantly, the diffusion coefficients of the signals verify that the object being detected is ˜100 nm in size, which corresponds with the size of an Ab-bound virion. Thus, FCS generates an unalloyed profile of virion immunoreactivity.

In some embodiments, an analytical instrument is used that can accommodate 96 or 384 samples in a plate-based detection mode. In some embodiments, the instrument used has a footprint that is sufficiently small to allow operation under containment and/or in a clinical setting. In some embodiments, multiple samples can be placed in cover slides with sealed silicone chambers, chambered slides, or glass-bottom 96- or 384-well plates. Sample volumes in silicone chambers or 384 well plates can be in a range from about 5 to 10 μl. Samples can be transferred onto the chamber on cover glass or glass bottom well plate using a micropipette. The FCS-based analyses described herein requires that the drugs, e.g., antibodies, are fluorophore-labeled and introduced to viremic samples. It should be appreciated by the person skilled in the art that, in some embodiments, a saturating concentration of the at least one drug is introduced to the viremic samples so that all viruses that can bind to the at least one drug (e.g., because they're sensitive to the drug), have drug molecules to bind to. As each virion ventures into the ˜1 femtoliter focal volume of the instrument, detector channels register signal “bursts” from bound drugs, e.g., bound antibodies. In some embodiments, test conditions are such that the focal volume typically contains <1 particle at any given time. Drug-virion interactions are detected by FCS using up to four excitation wavelengths (488, 561, 635 and 730 nm) to excite the fluorophores in four distinct spectral regions (500-550 nm, 575-625 nm, 650-720 nm and 740-800 nm, respectively) through a 60× water objective. In some embodiments, sample plates can be moved via a computer-controlled stage for automated acquisition; an irrigation system ensures that water is present under the objective. In some embodiments, a high-performance computer controls the instrument, performs data acquisition and processes the data. In some embodiments, virion burst counting is performed using software and algorithms. In some embodiments, virion burst counting is performed using ISS Vistavision pattern recognition software and algorithms (ISS, Inc., Champaign, IL), which separate bnAb-bound-virion bursts from free bnAb signals and background noise and reconstructs the total number of bnAb-bound-virion present in the FCS observation volume [I. Altamore, 2013]. Burst counts can be simultaneously collected in multiple channels to determine single drug binding to virions (signals in one dedicated detector) or concurrent binding of several bnAbs by cross-correlation (multi-color) analyses of multi-channel signals. In some embodiments, the instrument reports absolute counts in each channel as well as virions/ml.

In some embodiments, virus-targeted drugs (e.g., bnAbs) are directly labeled with fluorescent dyes and mixed with a fluid sample comprising virions. The sample is placed in the instrument, which detects fluorescent events (i.e., bursts) in an observation volume comprising individual drug-containing or drug-bound virions using FCS. Computational algorithms/software quantify the number of virion bursts passing through the observation volume that have a diffusion coefficient substantially consistent with the size of the virion target. In some embodiments, the size of the virion target is about 100 nm. In some embodiments, all reactants are continuously in solution. In some embodiments, no washing steps or adsorption steps are necessary to enable the analyses. In some embodiments, the total number of drug-containing/bound virions is quantified, which is compared to the total population size to determine the fraction sensitive or resistant to the drug, e.g., bnAb, and hence the IIP value. In some embodiments, the total population size is determined using the platform by using probes that generically recognize the virions of interest, as understood by the person skilled in the art. For example, in one embodiment, the total population size is determined by quantifying viral RNA or DNA (for HIV 2 RNA=1 virus) or counting virions using a fluorescent probe that recognizes a surface marker presented by all virions. It should be appreciated by the skilled artisan that the nature of the surface marker must be tailored to the virus type of interest. For example, with HIV, the surface marker can be ICAM-1. In some embodiments, the total population size is determined using RT qPCR (Quantitative reverse transcription polymerase chain reaction). Advantageously, analytical times may be 1 to 30 minutes per sample.

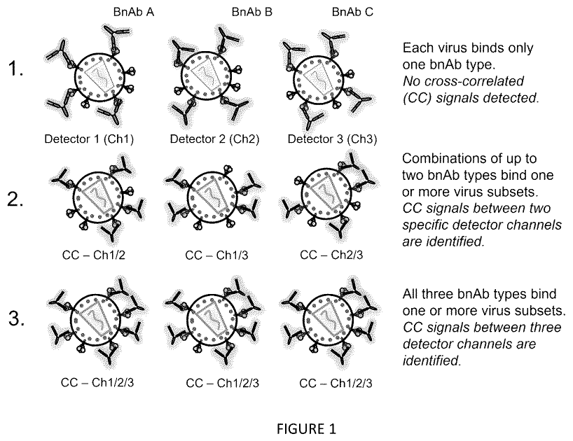

Advantageously, FCS allows multiplex detection of bnAb sensitivity or resistance in a single sample. Specifically, one test procedure can determine the proportions, or IIP value, of virions in a sample that are ignored by all members of a bnAb combination; are selectively covered by only certain bnAbs; or are concurrently covered by multiple bnAbs. This capacity allows assays to economize sample usage yet maximize analytical speed and informative value. A mixture of bnAbs, each conjugated to a different fluorophore, is reacted with sample virions. As each virion is detected, bound antibodies register fluorophore-specific signals in dedicated detector channels. A single virion bound by a single bnAb will appear as a burst only in the detector channel dedicated to the fluorophore bound to the single bnAb. Multiple bnAbs bound to a single virion (see, e.g., FIG. 1) will be detected as cross-correlated signal bursts in multiple channels (K. Ray, 2019; U. Kettling, 1998; K. G. Heinze, 2004]. Details regarding cross-correlation detection can be found, for example, in K. Ray, 2019. Subtracting the numbers of bnAb-bound virions of any sort from the entire virion population size reveals the proportion that is insensitive, i.e., resistant, to any bnAb action. In some embodiments, software and instrumentation capacities support multiplex analyses using up to two bnAbs. In some embodiments, software and instrumentation capacities support multiplex analyses using up to three bnAbs. In some embodiments, software and instrumentation capacities support multiplex analyses using up to four bnAbs. In some embodiments, software and instrumentation capacities support multiplex analyses using up to five bnAbs.

Accordingly, in some embodiments, a single drug can be tested to estimate efficacy in a single sample. In some embodiments, multiple drugs, e.g., two or three or four or five or more in a combination, can be tested simultaneously to estimate combination efficacy in a single sample. Each drug is labeled with a different fluorophore and simultaneously reacted with sample virions. Multiple detectors, each detecting a specific fluorophore-drug combination can be used to quantify targeted virion bursts. Cross-correlation of different signals for each virion burst reveals binding of virions by multiple drugs. In some embodiments, an IIP value, or log(fₐ/fᵤ) value, >about 3 evidences efficacy of the drug(s) towards the sample. In some embodiments, an IIP value, or log(fₐ/fᵤ) value, >about 4 evidences efficacy of the drug(s) towards the sample. In some embodiments, an IIP value, or log(fₐ/fᵤ) value, >about 5 evidences efficacy of the drug(s) towards the sample. In some embodiments, an IIP value, or log(fₐ/fᵤ) value, >about 6 evidences efficacy of the drug(s) towards the sample. In some embodiments, an IIP value, or log(fₐ/fᵤ) value, >about 7 evidences efficacy of the drug(s) towards the sample. In some embodiments, an IIP value, or log(fₐ/fᵤ) value, >about 8 evidences efficacy of the drug(s) towards the sample.

In some embodiments, dyes can be selected to enable fluorescent resonant energy transfer (FRET) signals of multiple drugs bound to individual virions. FCS in combination with FRET can be used to detect if two or more drugs, conjugated to their respective dyes, bind to a single target protein on a virion surface. Dual-FRET can be used to detect if three different drugs, conjugated to their respective dyes, bind to a single target protein on a virion surface.

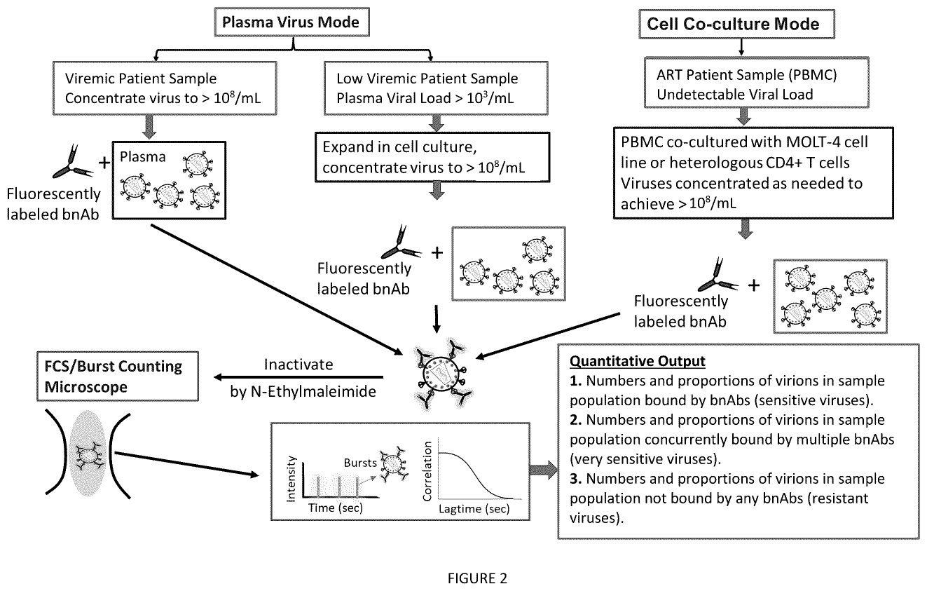

In some embodiments, an FCS-based bnAb resistance assay, as described in FIG. 2, is described for routine clinical use and data collection. Two test modes are introduced in FIG. 2, one for direct analysis of plasma viruses (the “plasma virus” mode), the second for analyzing latent viruses emerging in outgrowth co-cultures of peripheral blood mononuclear cells (PBMCs) from aviremic subjects (the “cell co-culture mode”). The “plasma virus” mode applies to plasma samples from viremic individuals (e.g., STI participants with rebounding viral loads or participants with breakthrough infections in prevention trials). In cases where viral loads are high (≥10⁶ copies/ml), viruses are concentrated ˜100-fold, for example by PEG-it™/centrifugation method. In samples with lower RNA copies (e.g., about 10³ copies/ml), virus is expanded in activated CD4+ T cell cultures, if possible, and concentrated as above. The fluorescent-labeled bnAbs are added to the concentrated virions and N-Ethylmaleimide (NEM) inactivated [D. R. Morcock, 2005] for FCS detection. The inactivation procedure maximizes safety during instrument manipulation. The numbers of virions bound by one or more bnAbs is determined by signals and/or cross-correlated signals in multiple detectors; these values are compared to the total virion population (RNA copy numbers) to determine the fractional sizes of bound versus unbound subpopulations and the IIP value.

In some embodiments, another FCS-based bnAb resistance assay, as described in FIG. 2, is described for routine clinical use and data collection. The second mode (“cell co-culture mode”) applies to samples from virus-suppressed individuals on ART (e.g., volunteers for STI therapy trials) who have little or no detectable plasma virus. This mode is used to assess risk of bnAb resistance and outgrowth by variants in viral reservoirs. Here, as shown in FIG. 2, activated PBMC samples are placed in co-culture with cell lines (e.g., MOLT-4) or heterologous CD4+ T cells to recover viruses via standard viral outgrowth methods routinely employed to assess viral reservoirs and latent viruses [G. M. Laird, 2016; J. C. Lorenzi, 2016]. Virions in culture supernatants will be concentrated, inactivated, and evaluated by FCS and/or cross-correlated signals to determine the fractions of bnAb-bound and unbound virions in the total population and the IIP value. The fractions are determined versus denominator values derived from viral RNA copy numbers or from counts of fluorescent protein markers introduced to all virion surfaces during replication in co-culture.

Advantageously, both embodiments (i.e., plasma virus mode and cell co-culture mode) allow multiplex analyses of up to three or more bnAbs per sample to assess HIV resistance/sensitivity.

In some embodiments, FCS-based drug resistance analyses only requires that the drugs, e.g., bnAbs, are fluorophore-labeled and introduced to viremic samples. As each virion ventures into the ˜1 femtoliter focal volume, detector channels register signal “bursts” from bound bnAbs. Test conditions are such that the focal volume typically contains ≤1 particle at any given time, each producing a signature fluorescent burst. Virion bursts are selected after autocorrelation analyses of whole fluctuation data and each 10 sec time increment indicates the signal diffusion coefficient corresponds to a 100 nm particle [K. Ray, 2014; R. Rigler, 2001]. Fitting the autocorrelation data to a 3D-diffusion model yields the translation diffusion time of a retrovirion (˜4 msec). The quantity and concentration of virions in a sample is determined from the number of detected bursts within a defined volume and measurement time frame, accounting for the translational diffusion time of virions. In some embodiments, autocorrelation plots can be used to report the percentage of bnAb in virus bound form. Under standardized conditions, these values provide another way to monitor consistent assay and reagent performance and quality.

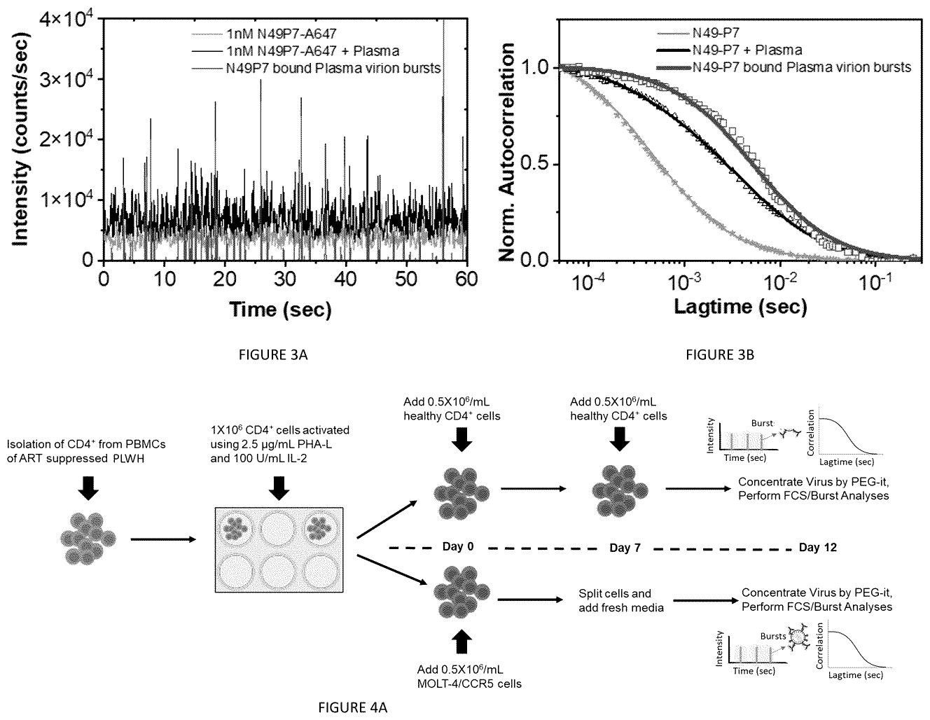

Additional advantages of the method described herein include, but are not limited to: (a) FCS techniques directly and quantitatively delimit bnAb-bound versus unbound subpopulations of virions in plasma or concentrated out of plasma or culture fluids (see, FIGS. 3 and 4). Up to three or more bnAbs (with dedicated labels) can be studied at once to comprehensively report bnAb coverage, susceptibility, or avoidance among the viruses harbored by a trial volunteer or participant; (b) Cross-correlated signals of multiple conjugated bnAbs register concurrent binding to single virions; (c) FCS techniques delimit bnAb-immunoreactive versus unreactive virus populations in virus outgrowth cultures from aviremic persons living with HIV (see, FIG. 4A). Although the co-culture step adds up to twelve days to the reporting time of the cell co-culture assay mode, FCS reporting of bnAb resistance in reservoir/outgrowth populations is more rapid than other approaches requiring multi-week efforts of Env sequencing, pseudovirus production and testing.

In some embodiments, FCS assays that allow simultaneous testing of samples for HIV sensitivity or resistance use only one bnAb. In some embodiments, FCS assays that allow simultaneous testing of samples for HIV sensitivity or resistance use two bnAbs. In some embodiments, FCS assays that allow simultaneous testing of samples for HIV sensitivity or resistance use three bnAbs. bnAbs that can be used in the FCS assays described herein include, but are not limited to, CD4bs (e.g., N49P7, VRC-01, VRC07-523LS or 3BNC117), V3 glycan (e.g., PGT121 or 10-1074), V2 apex (e.g., PGDM 1400 or CAP256-VRC26.25), MPER (e.g., 10E8 or DH511.2_K3), and any combination thereof. Although 10E8 may not be further considered for clinical use, bispecific antibodies based on 10E8 continue to see clinical development (e.g., NCT03875209). In some embodiments, the bnAbs combinations include 3BNC117, PGT121 and CAP256-VRC26.25. In some embodiments, the bnAbs combinations include 3BNC117, PGT121 and 10E8. In some embodiments, the bnAbs combinations include PGT121, CAP256-VRC26.25 and 10E8. In some embodiments, the bnAbs combinations include 3BNC117, CAP256-VRC26.25 and 10E8. In some embodiments, the bnAbs combination comprises anti-CD4bs N49P7. In some embodiments, the concentration of each bnAb is in a range from about 0.1-5 μg/ml. In some embodiments, the concentration of each bnAb is about 0.5 μg/ml. In some embodiments, the concentration of each bnAb is about 1 μg/ml. In some embodiments, the concentration of each bnAb is about 1.5 μg/ml. It can be envisioned by the skilled artisan that any new resistance test will have to accommodate the combinations of future bnAbs/bnAb classes for clinical testing.

In some embodiments, the anti-RSV mAb SYNAGIS® (palivizumab) can serve as negative control in the FCS experiments [K. Ray, 2014].

Accordingly, in one aspect, a method of calculating an Instantaneous Inhibition Potential (IIP) value of at least one drug in a fluid sample is described, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the IIP value of the at least one drug in the fluid sample, wherein the IIP value is the number of log 10 infections reduced by the at least one drug. In some embodiments, an IIP value>about 3 represents a fluid sample comprising a virus that is sensitive to the at least one drug. In some embodiments, an IIP value>about 4 represents a fluid sample comprising a virus that is sensitive to the at least one drug. In some embodiments, an IIP value>about 5 represents a fluid sample comprising a virus that is sensitive to the at least one drug. In some embodiments, an IIP value>about 6 represents a fluid sample comprising a virus that is sensitive to the at least one drug. In some embodiments, an IIP value>about 7 represents a fluid sample comprising a virus that is sensitive to the at least one drug. In some embodiments, an IIP value>about 8 represents a fluid sample comprising a virus that is sensitive to the at least one drug. In some embodiments, burst counts are simultaneously collected in multiple detector channels to determine concurrent binding of multiple drugs to a single virion by cross-correlation analyses of multi-channel signals, and multiple IIP values calculated.

In some embodiments, a method for quantifying a fraction of virions in a fluid sample that are sensitive to at least one drug is described, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the fraction of virions in the fluid sample that are sensitive to the at least one drug by calculating an Instantaneous Inhibition Potential (IIP) value of the at least one drug, wherein the IIP is an indication of the fractions of virions in the fluid sample that are sensitive to the at least one drug. In some embodiments, an IIP value>about 3 represents a fluid sample comprising a virus that is sensitive to the at least one drug. In some embodiments, an IIP value>about 4 represents a fluid sample comprising a virus that is sensitive to the at least one drug. In some embodiments, an IIP value>about 5 represents a fluid sample comprising a virus that is sensitive to the at least one drug. In some embodiments, an IIP value>about 6 represents a fluid sample comprising a virus that is sensitive to the at least one drug. In some embodiments, an IIP value>about 7 represents a fluid sample comprising a virus that is sensitive to the at least one drug. In some embodiments, an IIP value>about 8 represents a fluid sample comprising a virus that is sensitive to the at least one drug. In some embodiments, burst counts are simultaneously collected in multiple detector channels to determine concurrent binding of multiple drugs to a single virion by cross-correlation analyses of multi-channel signals.

In some embodiments, a method for quantifying a fraction of virions in a fluid sample that are resistant to at least one drug is also described, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the fraction of virions in the fluid sample that are resistant to the at least one drug by calculating an Instantaneous Inhibition Potential (IIP) value of the at least one drug, wherein the IIP is an indication of the fractions of virions in the fluid sample that are sensitive to the at least one drug. In some embodiments, an IIP<about 3 represents a fluid sample comprising a virus that is overly resistant to the at least one drug. In some embodiments, an IIP<about 4 represents a fluid sample comprising a virus that is overly resistant to the at least one drug. In some embodiments, an IIP<about 5 represents a fluid sample comprising a virus that is overly resistant to the at least one drug. In some embodiments, an IIP<about 6 represents a fluid sample comprising a virus that is overly resistant to the at least one drug. In some embodiments, an IIP<about 7 represents a fluid sample comprising a virus that is overly resistant to the at least one drug. In some embodiments, an IIP<about 8 represents a fluid sample comprising a virus that is overly resistant to the at least one drug. In some embodiments, burst counts are simultaneously collected in multiple detector channels to determine concurrent binding of multiple drugs to a single virion by cross-correlation analyses of multi-channel signals.

In another aspect, a method of identifying if at least one drug will substantially eliminate virus replication and/or suppress viral load to levels below detection in a subject is described, said method comprising:
calculating the Instantaneous Inhibition Potential (IIP) value of the at least one drug using a method described herein,
wherein if the calculated IIP value is greater than a threshold IIP determined for a known, efficacious drug, the virions are sensitive to the at least one drug, and said drug will likely substantially eliminate virus replication and/or suppress viral load to levels below detection. In some embodiments, the threshold IIP determined for a known, efficacious drug is about 3. In some embodiments, the threshold IIP determined for a known, efficacious drug is about 4. In some embodiments, the threshold IIP determined for a known, efficacious drug is about 5. In some embodiments, the threshold IIP determined for a known, efficacious drug is about 6. In some embodiments, the threshold IIP determined for a known, efficacious drug is about 7. In some embodiments, the threshold IIP determined for a known, efficacious drug is about 8.

In yet another aspect, a method of identifying, prior to treatment, at least one drug that extensively covers wild-type virus populations in a subject is described, said method comprising:
calculating the Instantaneous Inhibition Potential (IIP) value of the at least one drug using a method described herein, wherein if the calculated IIP value is greater than a threshold IIP determined for a known, efficacious drug, the virions are sensitive to the at least one drug, and said at least one drug will likely cover the virus in said subject. In some embodiments, the threshold IIP determined for a known, efficacious drug is about 3. In some embodiments, the threshold IIP determined for a known, efficacious drug is about 4. In some embodiments, the threshold IIP determined for a known, efficacious drug is about 5. In some embodiments, the threshold IIP determined for a known, efficacious drug is about 6. In some embodiments, the threshold IIP determined for a known, efficacious drug is about 7. In some embodiments, the threshold IIP determined for a known, efficacious drug is about 8.

In still another aspect, a method of identifying emerging endogenous bnAb resistance after exogenous bnAb treatment of a subject is described, said method comprising:
calculating the Instantaneous Inhibition Potential (IIP) value, in temporal samples, of the bnAb using a method described herein, wherein if the IIP value decreases in the temporal samples, the virions are becoming resistant to the bnAb. In some embodiments, if the IIP value is less than about 3, the virions are resistant to the bnAb.

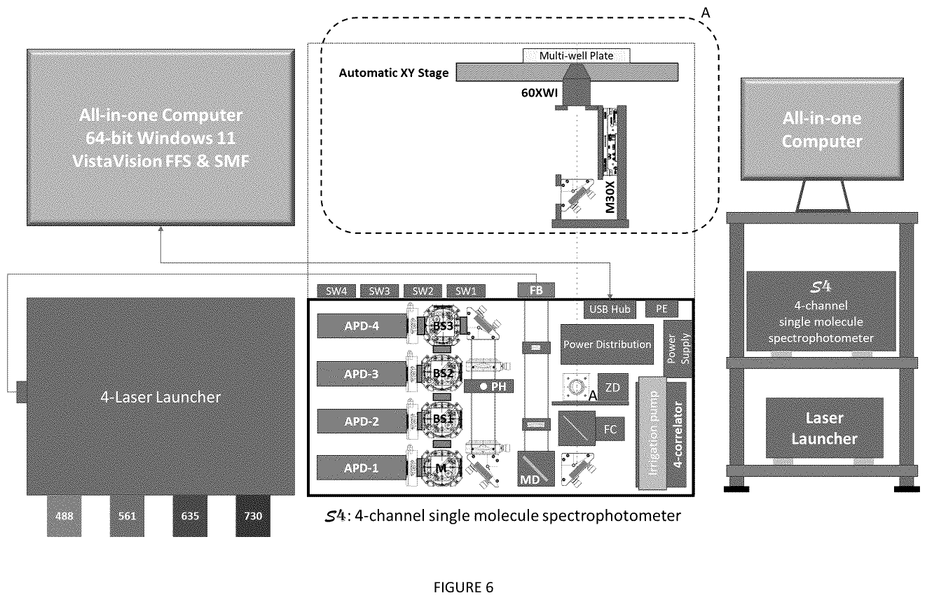

In another aspect, an instrument for FCS-based virion binding assays is described. A schematic of the instrument is shown in FIG. 6, comprising a four-channel single molecule spectrophotometer, a plate-based moving stage, a laser launcher, and a computing device. In some embodiments, the instrument is capable of multiplex, high-throughput collection and management of fluorescent signals at different spectral wavelengths. The instrument can detect fluorescently labeled bnAbs, bnAb-virion complexes or fluorescent virions by FCS using cover slides with sealed silicone chambers, chambered slides, or glass-bottom 96- or 384-well plates. Sample volumes in silicone chambers or 384 well plates can be 5 to 10 μl. Up to four excitation wavelengths (488, 561, 635 and 730 nm) can be utilized to excite the fluorophores in four distinct spectral regions (500-550 nm, 575-625 nm, 650-720 nm and 740-800 nm, respectively) through a 60× water objective. Sample plates will be moved via a computer-controlled plate-based moving stage for automated acquisition; an irrigation system ensures that water is present under the objective. The instrument can be constructed such that fluorescence signals are detected using combinations of high quality dichroic and bandpass filters to four cooled single photon counting avalanche photodiodes (SPADs); their output is sent to a 4-channel correlator. A high-performance computer controls the instrument, performs data acquisition and processing. In some embodiments, virion burst counting is accomplished using, for example, the ISS Vistavision pattern recognition software and algorithms, which separates bnAb-bound-virion bursts from free bnAb signals and background noise and reconstructs the total number of bnAb-bound-virion present in the FCS observation volume [I. Altamore et al., 2013]. In some embodiments, the bnAb thresholds can be set by including “bnAb only” wells/slides in each assay. Burst counts will be simultaneously collected in multiple channels to determine single bnAb binding to a single virion (signals in one dedicated detector) or concurrent binding of multiple bnAbs to a single virion by cross-correlation (multi-color) analyses of multi-channel signals. The instrument will report absolute counts in each channel as well as virions/ml.

Accordingly, in another embodiment, an apparatus for detecting interacting drug-virion bursts at a single virion level in an observation volume is described, said apparatus comprising:
a multi-laser launcher;
a plate-based moving stage;
a multi-channel single molecule spectrophotometer; and
a computing device.

In still another embodiment, a method of calculating an Instantaneous Inhibition Potential (IIP) value of at least one drug in a fluid sample using an apparatus, wherein the apparatus comprises a multi-laser launcher; a plate-based moving stage; a multi-channel single molecule spectrophotometer; and a computing device, is described, said method comprising:
mixing the fluid sample with at least one fluorescent-labeled drug conjugate to encourage drug-virion interactions in the mixture;
introducing the mixture to a sample location in the spectrophotometer;
using the multi-laser launcher to excite the fluorophores in the mixture;
using the spectrophotometer to detect interacting drug-virion bursts at a single virion level in an observation volume;
counting bursts in at least one detector channel using an algorithm;
determining a total population size of virions in the fluid sample; and
calculating the IIP value of the at least one drug in the fluid sample, wherein the IIP value is the number of log 10 infections reduced by the at least one drug.

In some embodiments, burst counts are simultaneously collected in multiple detector channels to determine concurrent binding of multiple drugs to a single virion by cross-correlation analyses of multi-channel signals, and multiple IIP values calculated.

In still another embodiment, a method of quantifying a fraction of virions in a fluid sample that are sensitive to at least one drug using an apparatus, wherein the apparatus comprises a multi-laser launcher; a plate-based moving stage; a multi-channel single molecule spectrophotometer; and a computing device, is described, said method comprising:
mixing the fluid sample with at least one fluorescent-labeled drug conjugate to encourage drug-virion interactions in the mixture;
introducing the mixture to a sample location in the spectrophotometer;
using the multi-laser launcher to excite the fluorophores in the mixture;
using the spectrophotometer to detect interacting drug-virion bursts at a single virion level in an observation volume;
counting bursts in at least one detector channel using an algorithm;
determining a total population size of virions in the fluid sample; and
calculating the fraction of virions in the fluid sample that are sensitive to the at least one drug by calculating an Instantaneous Inhibition Potential (IIP) value of the at least one drug,
   wherein the IIP is an indication of the fractions of virions in the fluid sample that are sensitive to the at least one drug.

In some embodiments, an IIP value>about 3 represents a fluid sample comprising a virus that is sensitive to the at least one drug. In some embodiments, an IIP value>about 4 represents a fluid sample comprising a virus that is sensitive to the at least one drug. In some embodiments, an IIP value>about 5 represents a fluid sample comprising a virus that is sensitive to the at least one drug. In some embodiments, an IIP value>about 6 represents a fluid sample comprising a virus that is sensitive to the at least one drug. In some embodiments, an IIP value>about 7 represents a fluid sample comprising a virus that is sensitive to the at least one drug. In some embodiments, an IIP value>about 8 represents a fluid sample comprising a virus that is sensitive to the at least one drug. In some embodiments, burst counts are simultaneously collected in multiple detector channels to determine concurrent binding of multiple drugs to a single virion by cross-correlation analyses of multi-channel signals. It should be appreciated by the person skilled in the art that this aspect can also be used to determine the fraction of virions in a fluid sample that are resistant to the at least one drug.

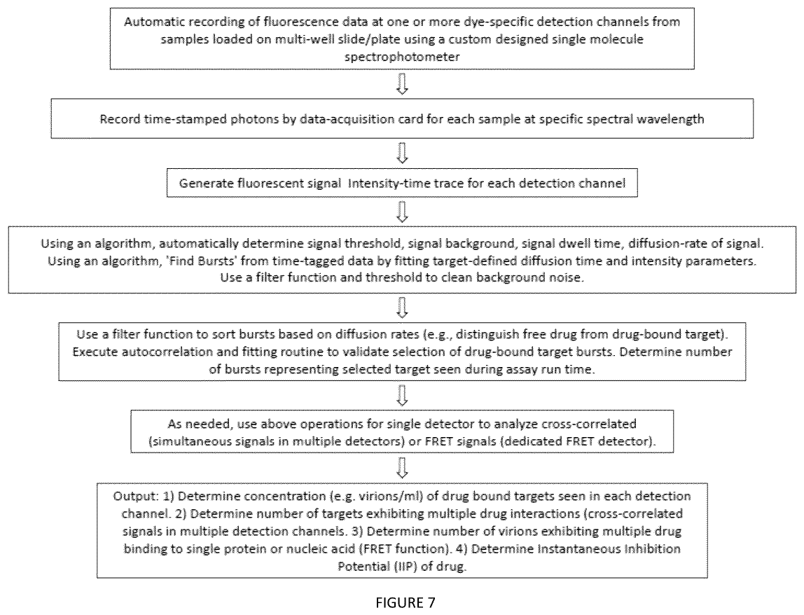

In some embodiments, the instrument is controlled by software and the data collected is analyzed using computational algorithms/software to determine if virions in a fluid sample are sensitive or resistant to the at least one drug. In some embodiments, the computational algorithms/software described herein perform the steps illustrated in the flowchart of FIG. 7. As shown in FIG. 7, in some embodiments, the algorithm/software follows the steps of: automatic recording of fluorescence data at one or more dye-specific detection channels from samples; record time-stamped photons by a data-acquisition card for each sample at a specific spectral wavelength; generate fluorescent signal intensity-time trace for each detection channel; using an algorithm, automatically determine signal threshold, signal background, signal dwell time, and diffusion-rate of the signal; using an algorithm, find bursts from time-tagged data by fitting target-defined diffusion time and intensity parameters; use a filter function and threshold to clean background noise; use a filter function to sort bursts based on diffusion rates (e.g., distinguish free drug from drug-bound target); execute autocorrelation and fitting routine to validate selection of drug-bound target bursts; determine the number of bursts representing selected target seen during assay run time; if it's necessary to analyze cross-correlated or FRET signals, use the same operations previously performed for a single detector; and calculate outputs including (1) the concentration (e.g., virions/mL) of drug-bound targets seen in each detection channel, (2) the number of targets exhibiting multiple drug interactions (i.e., cross-correlated signals in multiple detection channels), (3) the number of virions exhibiting multiple drug binding to single protein or nucleic acid (i.e., FRET function), and (4) determine IIP of the at least one drug.

Accordingly, in another aspect, a method of calculating an Instantaneous Inhibition Potential (IIP) value of a sample using computational software/algorithms is described, said method comprising: recording of fluorescence data of the sample for each dye-specific detection channel;
recording time-stamped photons by a data-acquisition card for the sample at a specific spectral wavelength;
generating fluorescent signal intensity-time traces for each dye-specific detection channel;
using an algorithm to determine signal threshold, signal background, signal dwell time, and diffusion-rate of the signal;
using an algorithm to identify bursts from time-tagged data by fitting target-defined diffusion time and intensity parameters;
using a filter function and threshold to clean background noise;
using a filter function to sort bursts based on diffusion rates;
executing an autocorrelation and fitting routine to validate selection of drug-bound target bursts;
determining the number of bursts representing selected targets seen during an assay run time;
optionally analyzing cross-correlated or FRET signals; and
calculating the IIP of the drug.

In some embodiments, this aspect can be used to calculate at least one additional output selected from (1) the concentration of drug-bound targets seen in each detection channel, (2) the number of targets exhibiting multiple drug interactions, if measured, and/or (3) the number of virions exhibiting multiple drug binding to single protein or nucleic acid, if measured. It should be appreciated by the person skilled in the art that this computational software/algorithm method can include optional steps, specifically the detection of a single drug binding to single targets, the detection of multiple drugs bound to single targets, and/or the detection of multiple drugs bound to single protein/nucleic acid on single virion.

The present subject matter may be a system, a method, instrument, and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The features and advantages of the invention are more fully shown by the illustrative examples discussed below.

### Example 1

A proof-of-concept experiment using a cryopreserved plasma sample from an HIV+subject with a high viral load (1.33×10⁷ RNA copies/ml) is shown in FIG. 3. The sample was probed with N49P7, a near pan-neutralizing bnAb against the CD4 binding site (CD4bs) [M. M. Sajadi, 2018] that supports FCS analyses [K. Ray et al., 2014; P. Agrawal, 2019]. In this case, N49P7 IgG was labeled with ALEXA FLUOR® 647 and added to undiluted plasma. Experimental conditions and parameters otherwise followed previously published studies [M Mengistu et al., 2015; K. Ray et al., 2019; K. Ray et al., 2014; P. Agrawal, 2019] For example, bnAbs were each labeled with a specific fluorophore, then reacted with virions in solution. As each virion ventures into the ˜1 femtoliter focal volume, detector channels register signal “bursts” from bound bnAbs. Test conditions are such that the focal volume typically contains≤1 particle at any given time, each producing a signature fluorescent burst. Virion-specific bursts are identified via autocorrelation analyses of whole fluctuation data, showing that the signal diffusion coefficient corresponds to a 100 nm particle. Fitting the autocorrelation data to a 3D-diffusion model verifies a translation diffusion time of a retrovirion (˜4 msec) and determines the number of virions/unit volume in a sample exhibiting a fluorescent bnAb binding signal. In such experiments, it is necessary to distinguish bound versus unbound bnAb fluorescence bursts. Making this distinction considers that unbound bnAbs have faster diffusion rates versus those bound to virions, and that virions are likely bound by >1 bnAb. Thus, the burst intensities of free bnAbs will on average be lower than those of bound virions because they present fewer fluorophores and exhibit a shorter dwell time in the detection window. Bursts from labeled bnAb alone were recorded over a 60 second period (see, FIG. 3A, green trace line) and used to conservatively set a threshold at two-times the highest burst intensity detected. Burst intensities above this threshold were taken as bnAb-bound virions (see, FIG. 3A, shown as bursts in red). This assumption was validated by autocorrelation plots of temporal fluorescence data within each 10 sec time window or the complete time frame. These measurements determined that the diffusion rates of each intensity peak above the threshold matched those of ˜100 nm diameter objects (see, FIG. 3B, scattered plot in red and fit to the data shown in solid red line). A modified version of burst analysis software that automatically calculates background and free bnAb thresholds in every experiment by an innovative pattern recognition (PR) algorithm was used (ISS Vistavision, ISS Inc., Champaign, IL, USA). The algorithm calculates the dwell time of each individual burst, which is a function of the source diffusion rate, also verified by auto- or cross-correlation analyses. This capacity provides additional verification that the counted burst emanates from a virion.

### Example 2

In another proof-of-concept experiment, PHA- and IL-2 stimulated CD4+ cells from an HIV+subject on ART (no detectable plasma viral load) were co-cultured with MOLT-4 cells according to published methods [G. M. Laird, 2016]. Briefly, the CD4+ cells from the PBMCs from an HIV+subject on ART were isolated using EASYSEP® Human CD4+ T-cell Enrichment Kit (STEMCELL Technologies Inc., Canada) and around 95% purity (confirmed by flow cytometry) was achieved. From the CD4+ cells, around 1×10⁶ CD4+ resting T-cells were activated using 100 U/mL of IL-2 and 2.5 μg/mL of PHA-L. The next day, the activated CD4+ cells were co-cultured with approximately 0.5×10⁶ MOLT-4/CCR5 cells for producing HIV virions. Every 4th day the culture was split, and the supernatant was collected for analyses. The RT qPCR results suggests that HIV RNA can be detected (6×10⁵ HIV RNA copies/mL) in culture supernatant on day 8, and increased to 2×10⁷ copies/mL on day 12. HIV p24 level was detected on day 8 and increased to 212 pg/ml at day 12. Virus was concentrated by PEG-it™ from conditioned media collected periodically. Viruses were tested by FCS for binding to bnAbs PGT121, N49P7, and CAP256-VRC26.25 after inactivation by NEM. The viral load in the concentrated sample was also determined by RT qPCR (8×10⁹/ml); this value served as the denominator for calculating bnAb coverage. As shown in FIG. 4B, among viruses harvested from 12-day cultures in MOLT-4, 15%, 51% and 88% of variants bound bnAbs PGT121, N49P7 and CAP256-VRC26.25, respectively. Samples collected at later time points (sixteen days) contained larger amounts of virus, but the fractions of bnAb bound virus remained steady (not shown) as no bnAb was present in the co-culture to exert selective pressure. From the standpoint of viral resistance, the test mode revealed that this donor harbors populations of viruses that are unrecognized (resistant) to all test bnAbs, particularly PGT121. Accordingly, the donor might be excluded from a trial of the test bnAbs due to risk of viral rebound.

### Example 3

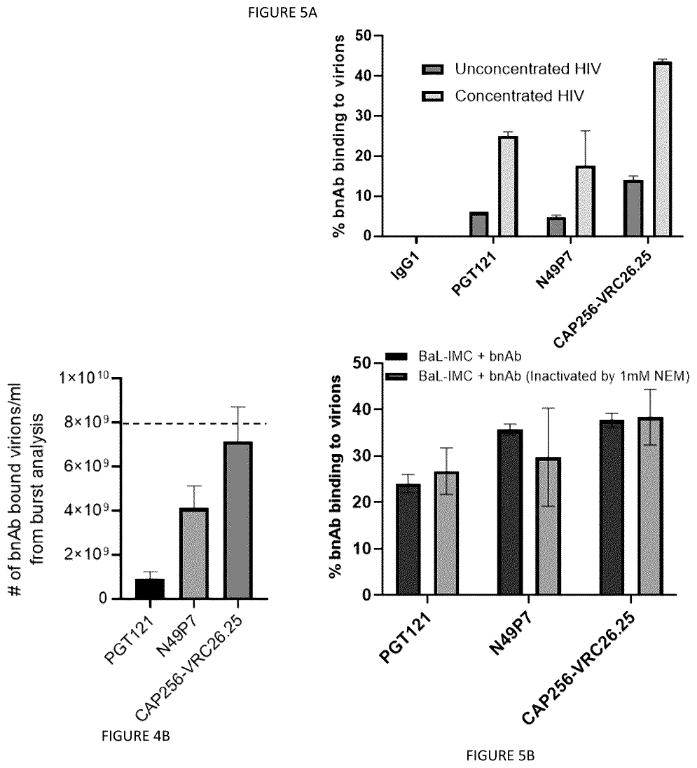

The proposed assay protocols (see, e.g., FIG. 2) rely on virions concentrated from plasma samples or culture fluids prior to analyses. This step facilitates more sensitive analyses when sample viral loads are lower, when a subject's circulating plasma antibodies risk confounding results, or when the subject is believed to harbor anti-bnAb antibodies due to treatment. In a preliminary study (see, FIG. 5A) it was determined that ˜80-fold concentration of virus (obtained by cell co-culture with MOLT-4) by PEG-it™ method does not alter binding patterns of bnAbs PGT121, N49P7, and CAP256-VRC26.25 (all tested at 0.15 μg/ml). Similarly, (see, e.g., FIG. 5B) NEM virus inactivation [D. R. Morcock, 2005] did not alter the relative bound populations of PGT121-, N49P7- and CAP256-VRC26.25-HIV BaL infectious molecular clone viruses.

### Example 4

In this example, the method of identifying bnAb combinations for FCS assays is described. Each bnAb combination have been tested (e.g., in five experiments each using triplicate measurements) for reactivity against a panel of well-characterized Env-pseudotyped viruses. Pseudoviruses are used because they can be expressed in a laboratory under stringent conditions and quality controls, thus minimizing caveats. The virus panel can include, but is not limited to, tier-1 Subtype B R5 HIV-1 BaL; tier-1 Subtype B X4 HIV-1 HXB2; tier-2 R5 Subtype B HIV-1 JRFL; and tier-2 Subtype C R5 viruses ZM282F PB18, ZM233M.PB6 and 1086c. The virus panel is chosen to establish the reproducible reactivity of bnAb combinations against viruses representing an array of epitope presentation patterns. For example, in some embodiments, HIV-1 BaL viruses are included as they should bind all bnAbs in the mixtures. In some embodiments, dedicated pseudovirus preparations, characterized according to gp120 and p24 antigen content and infectious titer are employed. The total number of virions in the preparations are determined by viral RNA copies and by FCS with dye-conjugated sCD4. Using three signal detectors, fluorescence bursts representing virus populations bound to one or more bnAbs (cross-correlated signals in multiple detectors; see, e.g., FIG. 1) are counted using the ISS Vistavision software described above, which sets the background burst thresholds for free bnAbs and verifies that the dwell time of any burst above background is consistent with the diffusion of retroviral particles. In each test, burst counts are collected for one to ten minutes. All data are examined for inter-assay variability in bnAb binding profiles and binding efficiencies. Autocorrelation plots are fitted to a 3D-diffusion model to verify that detected signals have diffusion rates consistent with HIV virions. Combinations exhibiting significant inter-assay qualitative differences in bnAb binding profiles and/or no significant variances in quantitative measures (binding efficiencies, burst counts of signals) will be eliminated from further use.

### Example 5

### Detection Assay for Virion Susceptibility to mAbs in Culture Fluids

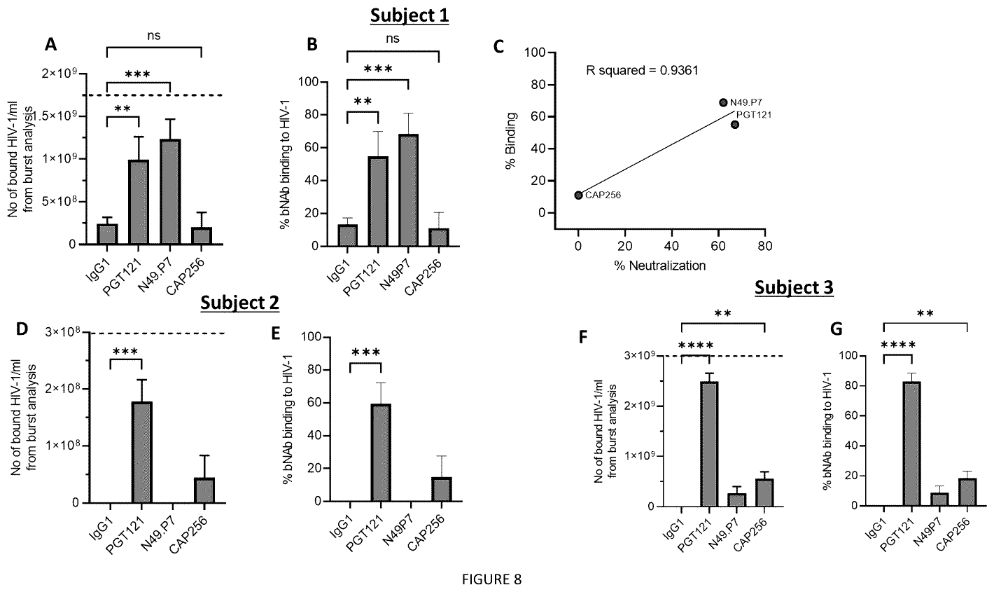

The FCS and burst counting method were applied to detect the different types of bnAb reactivity to wild type viruses from T cells in culture. PHA- and IL-2 stimulated CD4+ cells from HIV+ subjects on ART were co-cultured with MOLT-4 cells according to published methods (e.g., FIG. 4A). Viruses were concentrated from the conditioned media collected periodically and tested by FCS for binding to bnAbs PGT121, N49P7, or CAP256-VRC26.25. The viral load in the Day 12 concentrated sample was determined; this value served as a denominator for the bnAb coverage. Among viruses harvested from twelve-day co-cultures, the number of viruses bound to bnAbs PGT121, N49P7 or CAP256-VRC26.25 were determined to be distinctly lower than the viral load, suggesting all three donors harbor populations of viruses that are unrecognized (resistant) to all test bnAbs (FIGS. 8A-8G). All experiments were repeated three times, and average values are shown. Error bars indicate standard deviations from triplicate measurements. Control tests with nonspecific IgG1 produced signals with diffusion rates corresponding only to free antibody. The experiments were repeated two times with similar results. Overall, the data illustrated how FCS burst counting distinguishes and quantifies bnAb-bound subpopulations under various conditions.

### Example 6

### Analyzing Multiple mAbs Binding to Individual Virions and Virion Populations by Cross-Correlation Analyses

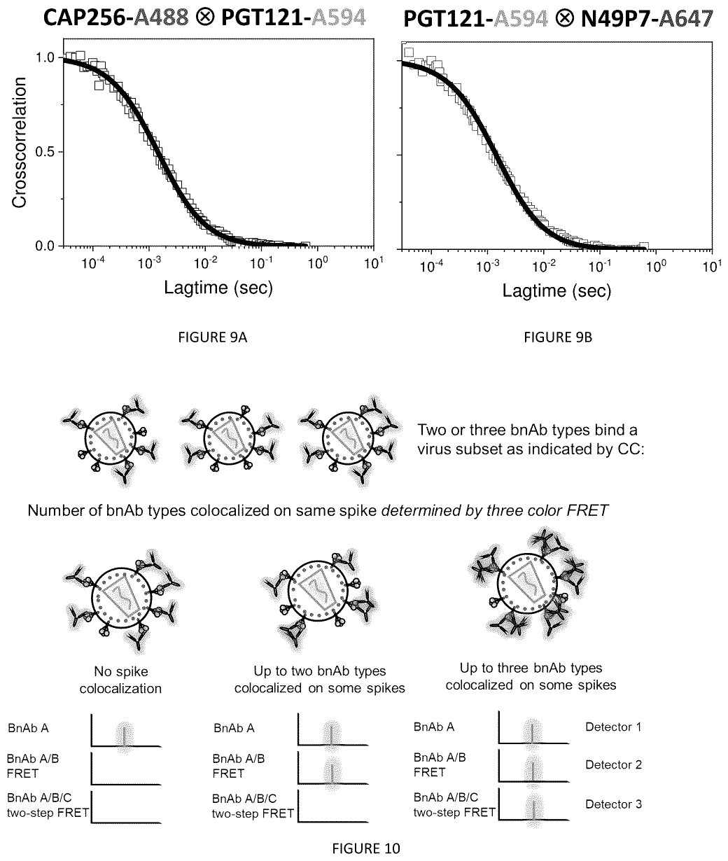

By means of fluorescent labeled bnAbs and employing an innovative application of FCS, approaches have been developed to simultaneously count, in one multiplex assay, the numbers of virions in a virus population that are bound by one or more bnAb types or avoid recognition altogether because they are not covered. Accordingly, virions concurrently bound to multiple bnAb types can be counted by tracking cross-correlated binding signals. FIGS. 9A-9B shows cross-correlation experiments with dual bnAb classes and HIV+plasma viruses revealing concurrent virion binding of CAP256-VRC26.25-A488 (anti-V2 apex)+PGT121-A594 (anti-V3 glycan) (FIG. 9A) or PGT121-A594+N49P7-A647 (anti-CD4bs) (FIG. 9B).

### Example 7

### FCS-FRET and Dual FCS-FRET Detection of Concurrent bnAb Binding to HIV Virions

FCS in combination with FRET was developed to detect if two or more bnAbs, conjugated to their respective dyes, bind to a single target protein on a virion surface (e.g., FIG. 10).

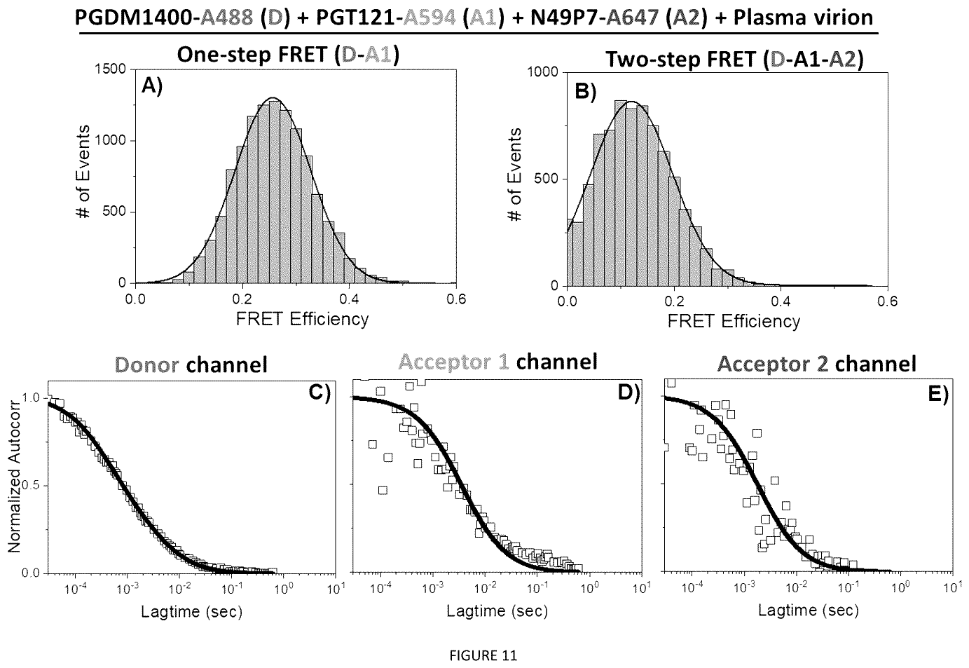

Dual-FRET was used to detect if bnAbs with three different specificities (e.g., PGDM1400, PGT121 and N49P7), conjugated to their respective dyes, bind to a single target protein on a virion surface. In the studies of dual FCS-FRET (FIGS. 11A-11B), HIV+plasma was reacted with conjugated whole bnAb IgGs: donor (A488 “D”) PGDM1400; acceptor 1 (A594 “A1”) PGT121; and acceptor 2 (A647 “A2”) N49P7. The fluorophore choices are set such that D cannot directly activate A2. The first step FRET (D-A1; FIG. 11A) fit a Gaussian profile with a mean efficiency of ˜25% while the mean dual FRET (D-A1-A2; FIG. 11B) efficiency was as expected lower at ˜12%. Importantly, FRET signals from both acceptor fluorophores (FIGS. 5D-5E) followed a diffusion coefficient of 6 μm²/sec. The autocorrelation plot for the “D” channel fit a two species diffusion model (as both unbound and bound bnAb should be detected in solution) whereas channels for FRET and dual FRET fit only a single species of 6 μm²/sec (only the virion bound bnAbs should exhibit FRET). This data demonstrates that FCS-FRET and dual FRET can detect the concurrent binding of bnAbs to a virion Env trimer, expressed by wild type virions in plasma. All experiments were repeated three times with similar results. Control tests with nonspecific SYNAGIS® IgG produced signals with diffusion rates only corresponding to free IgG.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Agrawal P, et al. Stoichiometric Analyses of Soluble CD4 to Native-like HIV-1 Envelope by Single-Molecule Fluorescence Spectroscopy. Cell Rep. 2019; 29(1):176-86 e4. doi: 10.1016/j.celrep.2019.08.074.
Altamore I, et al. Dual channel detection of ultra low concentration of bacteria in real time by scanning FCS. Meas Sci Technol. 2013; 24(6):65702. doi: 10.1088/0957-0233/24/6/065702.
Heinze K G, et al. Triple-color coincidence analysis: one step further in following higher order molecular complex formation. Biophys J. 2004; 86(1 Pt 1):506-16. doi: 10.1016/S0006-3495(04)74129-6.
Kettling U, et al. Real-time enzyme kinetics monitored by dual-color fluorescence cross-correlation spectroscopy. Proc Natl Acad Sci USA. 1998; 95(4):1416-20. doi: 10.1073/pnas.95.4.1416.
Klein F, et al. HIV therapy by a combination of broadly neutralizing antibodies in humanized mice. Nature. 2012; 492(7427):118-22. doi: 10.1038/nature11604.
Kong R, et al. Improving neutralization potency and breadth by combining broadly reactive HIV-1 antibodies targeting major neutralization epitopes. J Virol. 2015; 89(5):2659-71. doi: 10.1128/JVI.03136-14.
Laird G M, et al. Measuring the Frequency of Latent HIV-1 in Resting CD4(+) T Cells Using a Limiting Dilution Coculture Assay. Methods Mol Biol. 2016; 1354:239-53. doi: 10.1007/978-1-4939-3046-3_16.
Lorenzi J C, et al. Paired quantitative and qualitative assessment of the replication-competent HIV-1 reservoir and comparison with integrated proviral DNA. Proc Natl Acad Sci USA. 2016; 113(49):E7908-E16. doi: 10.1073/pnas.1617789113.
Mendoza P, et al. Combination therapy with anti-HIV-1 antibodies maintains viral suppression. Nature. 2018; 561(7724):479-84. doi: 10.1038/s41586-018-0531-2.
Mengistu M, et al. Antigenic properties of the human immunodeficiency virus envelope glycoprotein gp120 on virions bound to target cells. PLoS Pathog. 2015; 11(3):e1004772. doi: 10.1371/journal.ppat.1004772.
Morcock D R, et al. Elimination of retroviral infectivity by N-ethylmaleimide with preservation of functional envelope glycoproteins. J Virol. 2005; 79(3):1533-42. doi: 10.1128/JVI.79.3.1533-1542.2005.
Ray K, et al. Antigenic properties of the HIV envelope on virions in solution. J Virol. 2014; 88(3):1795-808. Epub 2013/11/29. doi: 10.1128/JVI.03048-13.
Ray K, et al. Concurrent Exposure of Neutralizing and Non-neutralizing Epitopes on a Single HIV-1 Envelope Structure. Front Immunol. 2019; 10:1512. doi: 10.3389/fimmu.2019.01512.
Rigler R, Elson E S, editors. Fluorescence Correlation Spectroscopy. Theory and Applications. New York: Springer; 2001.
Sajadi M M, et al. Identification of Near-Pan-neutralizing Antibodies against HIV-1 by Deconvolution of Plasma Humoral Responses. Cell. 2018; 173(7):1783-95 e14. doi: 10.1016/j.cell.2018.03.061.
Schoofs T, et al. HIV-1 therapy with monoclonal antibody 3BNC117 elicits host immune responses against HIV-1. Science. 2016; 352(6288):997-1001. Epub 2016/05/21. doi: 10.1126/science.aaf0972.
Wagh K, et al. Optimal Combinations of Broadly Neutralizing Antibodies for Prevention and Treatment of HIV-1 Clade C Infection. PLoS Pathog. 2016; 12(3):e1005520. doi: 10.1371/journal.ppat.1005520.
Wagh K, et al. Potential of conventional & bispecific broadly neutralizing antibodies for prevention of HIV-1 subtype A, C & D infections. PLoS Pathog. 2018; 14(3):e1006860. doi: 10.1371/journal.ppat.1006860.

What is claimed is:

1. A method for quantifying a fraction of virions in a fluid sample that are sensitive to at least one drug, said method comprising:
mixing the fluid sample with at least one fluorescent-labeled drug conjugate to encourage drug-virion interactions;
using fluorescence correlation spectrometry (FCS) to detect interacting drug-virion bursts at a single virion level in an observation volume;
counting bursts in at least one detector channel;
determining a total population size of virions in the fluid sample; and
calculating the fraction of virions in the fluid sample that are sensitive to the at least one drug by calculating an Instantaneous Inhibition Potential (IIP) value of the at least one drug,
wherein the IIP is an indication of the fractions of virions in the fluid sample that are sensitive to the at least one drug.

2. The method of claim 1, wherein the fraction of virions that escape from interaction with the at least one drug are resistant to the drug.

3. The method of claim 1, wherein single drug binding to virions is identified by signals in one dedicated detector.

4. The method of claim 1, wherein concurrent binding of multiple drugs to virions is identified by cross-correlation analyses of multi-channel signals.

5. The method of claim 1, wherein the binding of two or more drugs to a single protein on a virion surface is detected using fluorescent resonant energy transfer (FRET).

6. The method of claim 1, wherein the determination of the total population size of virions comprises using a probe that generically recognizes the virions.

7. The method of claim 1, wherein the fluid sample comprises blood, plasma, serum, mucosal fluids, or cell cultures.

8. The method of claim 1, wherein the fluid sample is from an animal or a human.

9. The method of claim 1, wherein the fraction of virions in the fluid sample that are sensitive to two drugs is quantified, wherein at least two fluorescent-labeled drug conjugates are mixed with the fluid sample, and wherein each drug conjugate has a different fluorescent label.

10. The method of claim 1, wherein the fraction of virions in the fluid sample that are sensitive to three drugs is quantified, wherein at least three fluorescent-labeled drug conjugates are mixed with the fluid sample, and wherein each drug conjugate has a different fluorescent label.

11. The method of claim 1, wherein the drug is an antibody, an antiviral drug, a combination of antibodies, or a combination of antiviral drugs.

12. The method of claim 1, wherein the drug is a broadly neutralizing antibody (bnAb) or a combination of bnAbs.

13. The method of claim 1, wherein the fluid sample comprises a virus population selected from retroviruses, sarbecoviruses/coronaviruses, or influenza viruses, preferably human immunodeficiency virus (HIV).

14. The method of claim 1, wherein the burst counting is performed using at least one algorithm.

15. The method of claim 1, further comprising concentrating a virus population comprising the virions prior to mixing with the at least one fluorescent-labeled drug conjugate.

16. The method of claim 15, further comprising recovering viruses using viral outgrowth methods.

17. The method of claim 1, wherein the fluid sample comprises HIV and an IIP less than about 5 indicates that the virus is resistant to the at least one drug.

18. The method of claim 1, wherein the fluid sample comprises HIV and an IIP more than about 5 indicates that the virus is sensitive to the at least one drug.

19. The method of claim 1, wherein the IIP value is the number of log 10 infections reduced by the at least one drug.

20. A non-transitory computer-readable storage medium, storing a computer program thereon which, when run in a computer, causes the computer to carry out any of the steps of the method according to claim 1.

21. The non-transitory computer-readable storage medium of claim 19, comprising pattern recognition software and algorithms, which separate interacting drug-virion bursts from non-interacting drug signals and background noise and reconstructs the total number of interacting drug-virions present in the observation volume.

22. A method of identifying, prior to treatment, at least one drug that extensively covers wild-type virus populations in a subject, said method comprising:
calculating an IIP value of the at least one drug,
wherein if the calculated IIP value is greater than a threshold IIP determined for a known, efficacious drug, the virions are sensitive to the at least one drug, and said at least one drug will likely cover the virus in said subject.

23. A method of identifying emerging endogenous broadly neutralizing antibody (bnAb) resistance after exogenous bnAb treatment of a subject, said method comprising:
calculating an IIP value, in temporal samples, of the bnAb,
wherein if the IIP value decreases in the temporal samples, the virions are becoming resistant to the bnAb.
